(19) United States
(12) Patent Application Publication
(10) Pub. No.: US 2009/0148913 A1
(43) Pub. Date: Jun. 11, 2009

(54) Storage of Cellulosic Feedstocks to Facilitate Biofuel Production

(76) Inventor: Lawrence A. Shrawder, Kempton, PA (US)

Correspondence Address: MILLER LAW GROUP, PLLC, 25 STEVENS AVENUE, WEST LAWN, PA, 19609, US

(21) Appl. No.: 12/330,498

(22) Filed: Dec. 8, 2008

Related U.S. Application Data
(60) Provisional application No. 61/012,726, filed on Dec. 10, 2007.

(51) Int. Cl. C12P 19/14

(52) U.S. Cl. 435/99

(57) ABSTRACT

A method for storing cellulosic feedstock materials, particularly corn cobs, to facilitate the production of ethanol therefrom is effective to store feedstocks having a moisture content between about 20% and 50%. The method is initiated with the piling of the feedstock into a smooth, substantially rounded pile. The cellulosic biomass desirably has a substantially uniform average moisture content throughout the pile. High and low moisture materials can be mixed uniformly before being accumulated in the pile. The incorporation of oxygen into the pile is controlled by packing the pile in accordance with the moisture content and by covering the pile with an impermeable material. The pile of cellulosic feedstock achieves a temperature from 150 to 170° F., which depletes oxygen and creates acetic acid to facilitate subsequent processing of the feedstock into ethanol.

### CROSS-REFERENCE TO RELATED APPLICATIONS

This application claims priority on U.S. Provisional Patent Application Ser. No. 61/012,726, filed Dec. 10, 2007, entitled “Storage of Cellulosic Feedstocks to Facilitate Ethanol Production”, the content of which is incorporated herein by reference.

### FIELD OF THE INVENTION

The present invention relates generally to the storage of cellulosic feedstock materials, and more particularly, to the year-round storage of corn cobs and/or other cellulosic biomass containing from about 20 to about 50 percent moisture through the use of aerobic bacteria to create an anaerobic environment.

### BACKGROUND OF THE INVENTION

In recent years, ethanol production has moved to the forefront of alternative energy options due to the rise in the cost of petroleum based fuels. In the United States, commercial ethanol production is almost entirely based on cornstarch. While corn grain is relatively easy to convert to ethanol, an increased demand for corn has subsequently raised the price of corn and food products dependant on corn for production, such as meat and corn syrup. Any significant increase in ethanol production in the future will require feedstocks that are not part of the human food supply. One of the most promising feedstocks is cellulosic feedstocks, such as corn cobs. The utilization of corn cobs in the production of ethanol offers several advantages. For instance, corn cobs contain approximately 42 percent cellulose and 38 percent hemicellulose. These structural polysaccharides can be converted into simple sugars, principally glucose and xylose. These sugars, in turn, can be fermented into ethanol and other chemical products.

Additionally, corn cobs are readily available and are located in proximity to biorefineries that currently produce grain ethanol. Because of their greater bulk density, corn cobs are the most efficient part of the corn plant (excluding grain) to store and transport. This does not mean, however, that storing and transporting the cobs is either efficient or inexpensive. Corn cobs are produced once a year over a period of approximately eight weeks in the fall. Accordingly, in order for corn cobs to be an effective feedstock for the formation of ethanol, efficient cost-effective methods for storing corn cobs for year round use in ethanol production plants need to be developed.

It is well known in the art that forage crops as well as corn cobs can be stored safely provided the moisture content is less than 20 percent or over 50 percent. Examples of the safe storage of forage crops include hay that has a moisture content under 20 percent and silage that has a moisture content of over 50 percent.

In dry storage, as moisture levels fall below 20 percent, microorganisms that degrade biomass materials such as molds, fungi, and bacteria, become increasingly inactive. Most ligno-cellulosic feed stocks can be safely stored aerobically at 15 percent or less moisture for extended periods of time. Grain can be stored safely under 13 percent moisture. Forage crops, on the other hand, can be stored with a higher moisture content through the use of an acid treatment. With the addition of organic acids, principally propionic, formic, and citric acids, both forage and grain can be stored aerobically at room temperature up to approximately 25 percent moisture. This acid treatment lowers the pH to around 4.0, which combined with the low availability of moisture, allows safe aerobic storage of grain and hay.

Forage crops with a moisture content greater than about 50% are conventionally ensiled. In creating silage, however, a different mechanism of storage is used. With a moisture level between approximately 50 and 80 percent, microorganisms thrive. However, safe storage at these moisture levels can only be accomplished by providing an anaerobic environment with sufficient sugars so that fermentation bacteria can lower the pH through the production of organic acids, primarily lactic acid. When silage reaches a pH in the range of 3.8 to 4.2 in an anaerobic environment, microbial action generally ceases.

Storing forage crops or corn cobs in the moisture range from 20-50 percent is not desirable because of high dry matter losses, spoilage, and the risk of fire from uncontrolled microbial action that may lead to spontaneous combustion of the stored matter. For forage crops or corn cobs harvested between about 20 and about 50 percent moisture, the most viable options currently available for safe storage include: (1) drying the crop to below 15 percent moisture or (2) adding organic acids to lower the pH to between 3.8 and 4.2. While effective, both of these options are very expensive and the presence of excess organic acids (such as lactic acid) can exert an inhibitory effect and interfere with the microbial conversion of the cellulose and hemicellulose into simple sugars for conversion into ethanol. Corn cobs are generally harvested as a co-product when the corn is between 15 and 25 percent moisture. At this point cob moistures are generally 20 to 50 percent moisture. Accordingly, these existing methods are largely unsuitable for producing and storing low-cost feedstocks for the biofuel industry.

In addition to the problems associated with the moisture content of the stored feedstock, cellulosic feedstocks are very bulky and require huge volumes of space for storage. For example, to produce 100 million gallons of cellulosic ethanol per year, which is roughly the production of an average corn ethanol plant, the plant would require approximately 1.1 million tons of corn cobs. Such an amount of corn cobs would take up approximately 180 million cubic feet of storage, or approximately 205 acres of buildings that are full of cobs stacked 20 feet high. Clearly, indoor storage of corn cobs is not cost-effective.

Outdoor storage of corn cobs and other cellulosic biomass has its challenges and detributes as well. For instance, wind and rainfall can add unwanted water and/or oxygen to the storage piles, exacerbating dry matter loss, fire risk from heating, spoilage, and/or composting of the biomass. Recently, there have been several studies, such as one conducted by the American Society of Agricultural Engineers (ASAE) in 1986, to study methods of storing corn cobs. This study concluded that storing dry cobs (i.e., corn cobs containing less than about 20 percent moisture) outside in large piles was feasible. However, the study asserted that wet cobs stored in outdoor piles, even with forced ventilation, suffered prohibitively high loss rates. Thus, even among cob processors who stockpile dry seed cobs outside every year, it is believed that storing wet corn cobs is highly unlikely.

### SUMMARY OF THE INVENTION

It is an object of the present invention to provide an efficient, cost-effective, and safe storage of corn cobs and/or other cellulosic biomass that has a moisture content between about 20 to about 50 percent.

It is also an object of the present invention to provide for the safe storage of wet corn cobs and other cellulosic biomass in outdoor piles without the creation of excessive levels of organic acids or dry matter loss.

It is another object of the present invention to provide a suitable environment for the microbial conversion of sugars within cellulose and hemicellulose to cellulosic ethanol or other biofuels and biochemicals.

It is a further object of the present invention to create a storage environment that is suitable for the pre-treatment and hydrolysis of cellulose and hemicellulose into sugars and/or fermentation of the resultant sugars into ethanol or other biofuels and biochemicals.

It is still another object of this invention to provide a method of storing corn cobs and/or other cellulosic biomass materials in a manner that will facilitate the subsequent production of ethanol or other biofuels and biochemicals therefrom.

It is a feature of this invention that the cellulosic biomass storage method generates dilute organic acids that enhances the steam explosion technique for the pretreatment of cellulosic biomass materials to expose the cellulose to enzymes for the hydrolosis thereof.

It is an advantage of this invention that the storage process produces small amounts of organic acids from corn cobs when stored according to the disclosed method.

It is another advantage that the production of acetic acid can be utilized with the steam explosion method of pretreatment for enzymatic hydrolysis to facilitate ethanol production from a cellulosic biomass material.

It is another feature of this invention that the cellulosic biomass material has a moisture content between about 20% and about 50%.

It is yet another feature of this invention that the cellulosic biomass material is packed into a pile with a substantially uniform distribution of moisture throughout the pile.

It is still another advantage of this invention that the pile of cellulosic material can contain cellulosic material having varying moisture levels so long as the different moisture levels of the cellulosic biomass materials are at least substantially uniformly distributed within the pile.

It is still another feature of this invention that water can be added to cellulosic biomass materials that have a moisture content below about 25% to raise the overall average moisture content of the materials within the pile.

It is yet another feature of this invention that the storage method includes the step of accumulating biomass materials into a smooth, substantially uniformly shaped pile having a rounded top to shed rainwater.

It is still another feature of this invention that the pile of cellulosic biomass material is formed with an outer crust that will facilitate the shedding of water away from the pile and to restrict the passage of air into the pile.

It is yet another advantage of this invention that debris, such as corn husks and stalks can be mixed into the cellulosic biomass material placed on the outer surface of the pile to help form a crust on the pile.

It is a further advantage of this invention that the temperature of the pile of cellulosic biomass material can be monitored to assure that the pile achieves an internal temperature from about 110° F. to about 170° F.

It is still a further advantage of this invention that an impervious material can be applied to the outer surface of the pile of cellulosic biomass material to restrict the passage of air into the pile.

It is still another advantage of the present invention that an environment is created within the pile that is largely inhospitable to destructive microbes and which simultaneously causes a minimal lasting inhibitory effect on the beneficial microbes and/or enzymes used to convert the cellulose and hemicellulose into simple sugars.

It is a further advantage of the present invention that excessive levels of organic acids and dry matter losses are not created during the long-term storage of the corn cobs.

It is a further feature of the present invention that the creation and maintenance of an anaerobic or nearly anaerobic environment in the interior of the pile permits for the storage of corn cobs from one season to the next season.

These and other objects, features and advantages are accomplished according to the instant invention by a method for storing cellulosic feedstock materials, particularly corn cobs, to facilitate the production of ethanol, butynol and other biofuels and biochemicals therefrom. The feedstocks being stored have a moisture content between about 25% and about 50%. The method starts with the piling of corn cobs or other appropriate cellulosic biomass material into a smooth, substantially rounded pile having a width at the base of the pile approximately 2.5 times the height of the pile. The cellulosic biomass has a substantially uniform average moisture content throughout the pile such that high and low moisture materials can be mixed uniformly before being accumulated in the pile. Oxygen incorporated into the pile is controlled by packing the pile in accordance with the moisture content and particle size, or by covering the pile with a plastic material. The pile of cellulosic material achieves a temperature between 150° F. and 170° F. to deplete the oxygen and form acetic acid, which facilitates subsequent processing of the cellulosic feedstock to generate ethanol.

### BRIEF DESCRIPTION OF THE DRAWINGS

The advantages of this invention will be apparent upon consideration of the following detailed disclosure of the invention, especially when taken in conjunction with the accompanying drawings wherein:

FIG. 1 is a schematic illustration showing a cross-sectional view of a pile of cellulosic material formed according to the principles of the instant invention;

FIG. 2 is a schematic illustration showing an end elevational view of a pile of cellulosic material formed improperly;

FIG. 3 is a schematic illustration showing a cross-sectional view of a pile of cellulosic feedstock material formed according to an alternate configuration; and

FIG. 4 is a schematic illustration showing a cross-sectional view of a pile of cellulosic feedstock material formed according to another alternate configuration.

### DETAILED DESCRIPTION OF THE PREFERRED EMBODIMENT

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein. In the drawings, the thickness of the lines, layers, and regions may be exaggerated for clarity. It is to be noted that like numbers found throughout the figures denote like elements.

The present invention relates to the “wet storage” of corn cobs and other cellulosic biomass that have a moisture content from about 20 to about 50 percent. The cellulosic biomass is characterized by a high temperature deactivation of anaerobic Mesophilic and thermophilic microbes (including organic acid producing bacteria) and the anaerobic deactivation of aerobic microbes during the creation of a substantially anaerobic environment in piles of cellulosic material. As used herein, the term “biomass” is intended to denote living and recently dead biological material. The instant invention can be distinguished from dry storage, which is the aerobic storage of biomass and forage crops (usually undercover) that contain less than about 20 percent moisture content and ensiled storage, which is accomplished by the anaerobic covered storage of biomass and forage crops having between 50 and 80 percent moisture in the presence of microbially inhibitive quantities of organic acids generated at low temperatures (e.g. 90° F.) by anaerobic bacteria from the sugars inherent to the biomass. Although the storage of other biomass, forage crops, and other cellulosic material may be equally well applied to the present invention, the storage methods detailed herein will be made with reference to corn cobs.

Initially, corn cobs having similar characteristics such as, but not limited to, corn cobs that contain approximately the same moisture content and are similar in size and foreign matter content, are aggregated into piles having a certain shape (as described below). In a preferred embodiment, only an amount of oxygen sufficient to allow aerobic thermophilic bacteria to raise the temperature in the piles to between approximately 110° F. and approximately 160° F. is added to or incorporated into the piles. Desirably, the temperature in the piles is from about 140° F. to about 175° F., more desirably from about 150° F. to about 160° F., and most desirably, approximately 155° F. At no time should the temperature be permitted to exceed 180° F. At 180° F., the thermophilic bacteria within the pile cease to function and the possibility of a fire within the pile substantially increases.

Once the piles reach the desired temperature range, ideally, the incorporated oxygen is depleted and further infiltration of oxygen into the piles is reduced, if not stopped. During the heating process, carbon dioxide is produced during the degradation of the corn cobs as the respiration product of the aerobic thermophilic bacteria. As oxygen is depleted, the aerobic thermophiles become deactivated, thereby substantially stopping any further heating and/or dry matter losses. Further, it is to be appreciated that oxygen and water infiltration is desirably controlled to prevent reintroduction of undesirable microbes and to avoid additional excessive heating or risk of fire.

There are many interrelated variables involved in storing wet cobs, including, but not limited to, moisture content, cob particle size and uniformity, the presence of foreign matter such as husk and leaf material, the weather during the corn cob piling, the weather during the storage of the corn cobs, the shape of pile, the size of pile, the compression or density of pile, the oxygen content during piling, and the subsequent infiltration of water and oxygen after heating. For the purposes of this disclosure, these variables are broken down into the following categories.

Physical Properties of the Corn Cobs:

(1) Moisture Content

The initial moisture content of the corn cobs will determine the amount of oxygen necessary to provide the proper heating and subsequent storage of the pile. A high moisture content corresponding to the upper end of the moisture range (i.e., approximately 43 to 55 percent moisture) combined with too little oxygen can cause the pile not to reach sufficient temperatures to deactivate undesirable microbes with the resultant production of organic acids, such as acetic, lactic acid, and other organic acids. At the middle of the moisture range, (i.e., approximately 26 to 43 percent moisture), too much oxygen can promote excessive dry matter losses and/or fire. At the low end of the moisture range, (i.e., approximately 20 to 26 percent moisture, or below about 26% moisture), insufficient moisture and/or the addition of too much oxygen causes an inability to properly heat the piles, which results in losses due to consumption by yeasts, molds, actinomycetes, and other bacteria. The moisture content in these cobs can be raised by the substantially uniform incorporation of additional water during the formation of the pile.

It is to be appreciated, however, that highly disparate moisture levels in adjacent cob masses is highly correlated to the risk of fire. Thus, if cobs are used in the formation of the pile with greatly disparate moisture content, such as can occur when cobs from different sources are utilized, these cob particles need to be at least substantially, and preferably uniformly mixed so that the average moisture content of the corn cobs throughout the pile is substantially uniform. For example, cobs with less than 25% moisture content and cobs with greater than 50% moisture content can be utilized if these cobs having disparate moisture contents are properly mixed before or while they are being accumulated into a pile.

(2) Particle Size and Uniformity

At the upper end of the moisture range, a larger, more uniform particle size ensures sufficient oxygen levels in the piles for the corn cobs to properly heat. Moving from high to low moisture levels, the cob particle size and uniformity should decrease, thereby permitting less oxygen to be incorporated into the piles.

(3) Foreign Material

The incorporation of foreign material, such as leaves and husk has been generally thought to be undesirable when storing cobs. However, it has been discovered that moderate amounts of foreign material uniformly or substantially uniformly incorporated throughout the cob piles that fall within the middle moisture range and/or spread over the top of the piles in all of the moisture ranges can be beneficial in restricting both air and water infiltration into the pile during subsequent storage. The incorporated foreign material is preferably uniformly distributed throughout the pile so that vents or chimneys are not created in the pile. Such vents may allow an accumulation of condensed water vapor in the pile, creating a localized area of very high moisture, which in turn may result in the generation of heat greater than 180° F. and the ignition of adjacent lower moisture cobs.

Physical Characteristics of the Corn Cob Pile:

(1) Shape of the Pile

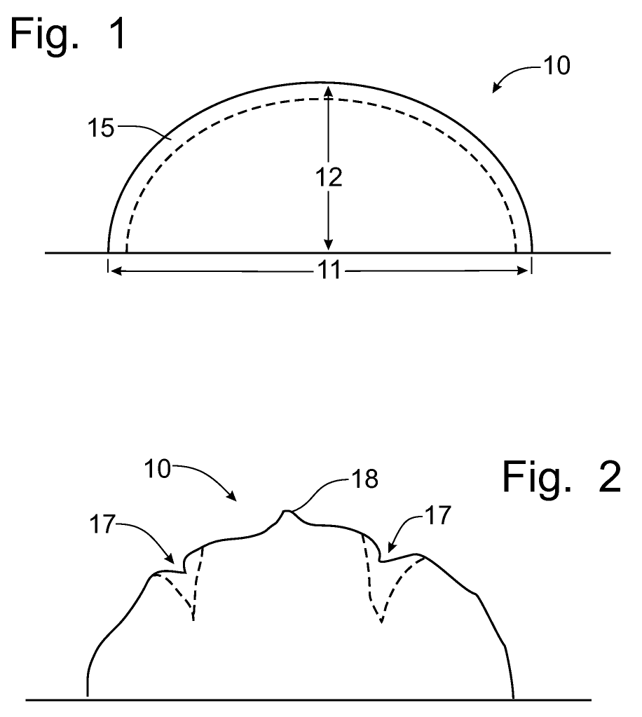

The shape of the pile is important for subsequent storage success. In a preferred embodiment as represented in FIG. 1, the pile 10 should be substantially smooth, generally without peaks or valleys, and mostly rounded on top with a base width 11 approximately 2.5 times the height dimension 12 of the pile 10. In exemplary embodiments, the length exceeds the width. Corn cobs are very absorbent, trapping rainfall in the outside crust 15 of the pile 10, and when the cobs are properly piled, the cobs are extremely efficient at shedding water. This water shedding capacity can be enhanced by the addition of cobs containing significant amounts of husk and leaf material over the top of pile after the pile 10 is formed. The addition of this foreign material creates a natural thatch similar to a thatched roof. However, any valleys 17 in the surface of the uncovered piles, as depicted in FIG. 2, will trap water, thereby creating concentrated areas of infiltration that over time can reactivate microbial action causing potential major dry matter losses as well as an increased potential for fire. Peaks 18, on the other hand, receive more oxygen infiltration than the surrounding areas, reactivating microbial action and causing steam vents or chimneys that can increase moisture content in localized areas, resulting in excessive dry matter losses and an increased potential for fire.

(2) Size of the Pile

The size of the pile is dependent upon the results expected, such the acceptable levels of loss in the piles, the moisture content, and the length of time that the corn cobs will be stored in the pile. Generally, in a properly built pile, a 6 to 9 inch saturated crust forms almost immediately, with an additional 1 to 2 inches of saturated crust forming per month, depending on rainfall amounts. The larger the pile, the lower the percentage of spoilage from the crust. It has been determined that smaller piles (e.g., approximately 100 to 400 tons) work well if the corn cobs are to be used relatively quickly (e.g., less than about 6 months). Larger piles (e.g., approximately 400 to 3,000 tons) work better if the cobs are to be stored for longer periods of time (i.e., 6 to 12 months). Smaller piles having approximately 300 to 400 tons per pile is a preferred embodiment due to the relative ease of covering the pile for better storage potential.

(3) Density of the Pile

Density is another variable that helps to control the oxygen content of the pile during heating. Generally, at the high end of the moisture range, if the cobs or chopped cob particle size is large enough, the pile can be packed. However, if uniformity in cob or particle size is lacking, excessive packing should be avoided. At midrange moistures, the corn cobs will generally accept significant packing. At the low end of the moisture range, packing is undesirable.

(4) Oxygen Content During Piling

The oxygen content within the corn cob piles is generally controlled by the moisture content of the corn cobs, the cob particle size and uniformity, foreign matter, and the degree to which the pile is packed. Ideally, sufficient oxygen should be incorporated into the pile so that aerobic, thermophilic bacteria can raise the temperature of the pile to approximately 110° F. to 155° F. degrees Fahrenheit. Ideally when the pile reaches about 155° F., the incorporated oxygen should be depleted and further infiltration of oxygen and water should cease. The efficiency of dry matter preservation is directly effected by the extent oxygen that is excluded at this point.

(5) Resistance to Infiltration of Water and Oxygen After Heating

After the cobs have been heated to the desired temperature by the thermophilic bacteria, additional water and oxygen should be restricted from infiltrating the pile. This can be easily accomplished by covering the pile with an impermeable, waterproof material, such as a plastic or vinyl sheet, after the pile is formed. Other means for restricting oxygen and water infiltration include manipulation of the cob size, the incorporation of foreign material, the pile shape, the size of the pile, and density of the pile as described supra.

Biological Characteristics and Processes

The inventive method promotes the heating of a corn cob pile to a temperature in the range from about 110° F. to about 160° F., preferably from about 150° F. to about 175° F., more preferably to about 150° F. to about 160° F., and most desirably, approximately 155° F., by aerobic, thermophilic bacteria. This rise in temperature is a distinctive departure from conventional silage formation, which is conducted at temperatures that are preferably under 90° F. By increasing the temperatures within the inventive piles to these higher temperatures, molds, yeasts and actinomycetes responsible for the deterioration of the corn cobs, as well as anaerobic organic acid producing bacteria such as the various strains of lactobacillus essential in the preservation of silage are deactivated or killed. More specifically, in the process of creating these higher temperatures, the thermophilic bacteria consume the oxygen, thereby causing the aerobic bacteria to begin to deactivate or die out as well and create an anaerobic environment within the interior of the pile.

During the process of making silage, both acetic acid and lactic acid producing anaerobic bacteria begin to convert plant sugars and starches into acetic and lactic acid. While these acids are being formed the pH of the silage is reduced first to 5.0, at which time the acetic acid producing bacteria deactivate. The lactic acid producing bacteria then take over and reduce the pH to approximately 4.0. At this pH all microbial activity within the pile ceases, ensuring safe storage of the silage. However, in the inventive method, when temperatures exceed 110° F., the lactobacillus bacteria die out and are no longer viable. The inventive method thus produces the desired result of prolonged storage of the corn cobs, with undetectable levels of lactic acid, butyric acid, and propionic acid. As a result, the inventive method does not substantially reduce the pH of the cobs. While small amounts of acetic acid are produced, it is usually 0.5 percent or less and can actually aid in the pretreatment process during the hydrolysis of the hemicellulose.

As long as this anaerobic environment is maintained, the corn cobs can be stored in the piles for relatively long periods of time (e.g., roughly up to one year). This hot (approximately 110° F. to 155° F.), anaerobic, and relatively low acid environment can generally be adapted to be reasonably tolerated by naturally occurring or genetically engineered mono- or multi-cultures of thermophilic anaerobic bacteria such as ethanol producing Clostridium thermocellum. While current microbes such as Clostridium thermocellum are relatively inefficient in converting biomass directly to ethanol, genetic engineering holds great promise for new microbes such as E. Coli to be adapted for useful ethanol production in this anaerobic environment.

The relatively inexpensive nature of the inventive storage method means that even less efficient microbes or enzymes can be economically permitted to take relatively long periods of time to convert the cellulose and hemicellulose into simple sugars, ethanol, and/or other biochemicals. In addition, as the ethanol level in the cellulosic biomass reaches levels that inhibit the life or function of these microbes, carbon dioxide or other gases non-reactive with the ethanol can be passed through these piles, collected, stripped of ethanol, and recycled through the pile to help maintain a microbe friendly environment. As thermophillic ethanolagens are discovered and/or engineered to operate at temperatures approaching 180° F., the ethanol vapors may simply be collected and condensed. The inventive method clearly produces an environment with many possibilities for the low cost production of biofuels and biochemicals.

The Storage Process:

With the above parameters in mind, the storage process begins with the preparation of the corn cobs for storage. The cobs are preferably crushed, typically by the combine at the time the corn kernels are removed from the cobs, into a random mix of different sizes of cob pieces, which are generally split and less than 3 inches in length. The use of the term “cobs” herein is meant to encompass both whole cobs and cob pieces. The cobs are collected and then accumulated into a pile placed at a well-drained site. The formation of piles of corn cobs will typically be outdoors. Therefore, the site must be prepared in a manner that will prevent or minimize the drainage of rainwater into the pile.

The moisture content of the cobs is an important factor. The storage process works well with the cobs that have an average moisture content from about 30% to about 45%, and even better with cobs having a moisture content from about 35% to about 45%. If cobs with a uniform higher moisture content are to be stored, larger cob piece sizes up to whole cobs may be required to entrain sufficient oxygen in the pile to promote heating. Also, if cobs having a significantly higher moisture level are going to be placed into the pile with cobs having a lower moisture content, the disparate moisture cob pieces need to be at least substantially uniformly mixed before being accumulated into the pile. If the overall moisture content is below the desired range, water can be uniformly added to the cobs as they are being placed into the pile to raise the average moisture content. Uniformity in the average moisture content of the cobs throughout the pile is needed to assure that the desired temperature within the pile is achieved and maintained so that the heat within the pile does not exceed approximately 180° F. and potentially start a fire within the pile.

The cob particles can be packed as necessary as they are being accumulated into the pile to increase the density of the cellulosic biomass material as a means of limiting the entrained oxygen within the pile to control the heating cycle. The dense packing also helps restrict the infiltration of air (oxygen) into the pile after the heating cycle in uncovered piles. As the pile is being formed to the desired size and shape, the surface of the pile should ideally be shaped into a smooth, rounded configuration devoid of peaks and valleys to assist in the shedding of rainwater.

After the pile is completed, the outer crust of the pile will form naturally and should not be disturbed. Alternatively, the formation of the outer crust can be facilitated by incorporating foreign matter, such as husks and leaves, in addition to the cob particles into the surface during pile formation. The outer crust is intended to harden naturally to provide an improved watershedding capability for the pile so that rainwater will be drained off of and away from the pile. With proper construction of the pile and the appropriate moisture content and oxygen entrainment, the cobs will enter into the heating cycle whereby the aerobic thermophilic bacteria raises the internal temperature of the interior of the pile to preferably about 150° F. to about 160° F. The temperature of the interior of the pile can be monitored with temperature probes. Once the pile temperature reaches approximately 110° F., the bacteria that produce lactic acid are killed off. With sufficient moisture and oxygen thermophilic aerobic bacteria will continue to raise the temperature to about 155° F., killing off any remaining mesophilic bacteria including the remaining organic acid producing bacteria. However, small amounts of acetic acid are still generated by the process.

Once the pile reaches about 155° F. the temperature stabilizes and the thermophiles continue to consume the cellulosic biomass until the moisture or oxygen is exhausted. Ideally, it is at this precise point in the heating cycle, i.e., when the temperature first reaches about 155° F., that the pile runs out of oxygen. When the oxygen has been depleted, the heating cycle will stop. At this point, carbon dioxide produced by the thermophiles will have replaced the oxygen creating an anaerobic environment that, if properly maintained, ensures successful storage of the cellulosic biomass material. Since corn cobs have great insulation capability, the interior temperature can remain elevated for months without any outside assistance.

Once the pile has attained a temperature of about 155° F., further infiltration of air into the pile is undesirable and can be substantially eliminated by covering the pile with a substantially impermeable material, such as plastic or vinyl, utilizing oxygen limiting structures, such as silos, or the formation of an outer crust to protect the interior of the pile. The plastic material can be placed over the outer surface of the pile at the time of formation, in which case the outer crust will not be needed to shed water from the surface of the pile. Corn cobs stored with this method of storage can be maintained in the pile in an anaerobic environment without substantial loss of cellulosic material for relatively long periods of time (e.g., roughly up to one year). When cobs are being stored for relatively short periods of time, the outer crust that forms on properly shaped and packed piles is sufficient to limit oxygen and water infiltration to the interior of the pile.

Further, the dilute amounts of acetic acid formed during this storage process facilitates the hydrolysis of the hemicellulose into simple sugars during the steam explosion method of pretreatment for ethanol production after the feedstock material has been removed from the pile and processed. Placing a plastic membrane over the pile is difficult to accomplish under certain circumstances, as the rounded surface of the pile tends to act as an air foil that causes the plastic membrane to rise off the pile. Accordingly, if a plastic membrane is used, particularly at the formation stage of the pile, care must be taken to assure that the plastic membrane remains in place.

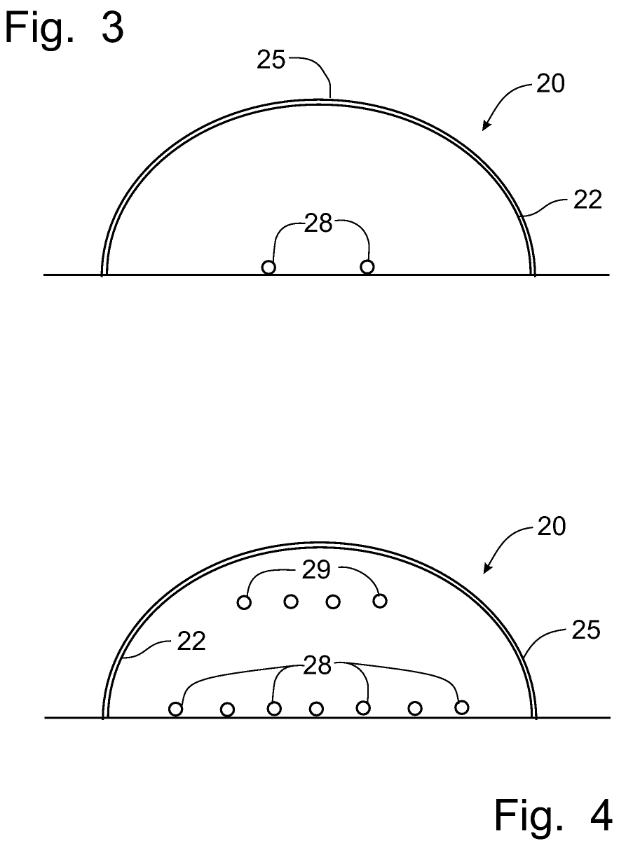

An alternative configuration for the formation of the pile 20 is depicted in FIG. 3. In this embodiment, a non-permeable, flexible material 25 is placed over the outer surface 22 of the pile 20 during the formation of the pile 20. Accordingly, infiltration of air and water into the pile is substantially eliminated. In this alternate embodiment, the average moisture content of the cellulosic feedstock forming the pile 20 should be within a preferred range of 35%-45% so that sufficient moisture is entrained within the pile 20 during formation thereof. The temperature of the pile 20 should be monitored through the use of temperature probes (not shown) to determine if the heating cycle is progressing appropriately. The pile 20 is preferably built over at least one perforated pipe 28, which can be used to introduce air into the interior of the pile 20 if the heating cycle does not produce the desired temperatures. The introduction of air (oxygen) into the interior of the pile would be undertaken only if the heating cycle does not generate temperatures in the 150-160° F. range, and care must be taken to see that the temperature within the pile does not exceed about 180° F. The pipes 28 can also be used to introduce a gas non-reactive with ethanol, such as carbon dioxide, to deactivate the aerobic bacteria by displacing oxygen within the interior of the pile 20 and slow down the heating cycle. This introduction of a non-volatile gas can be especially useful in limiting temperatures when creating lower temperature environments for certain specific ethanol producing bacteria.

As is depicted in FIG. 4, the pile 10 can be built with a first set of perforated pipes 28 along the bottom of the pile 10 and a second set of perforated pipes 29 near the top of the pile 10. The storage process defined herein, with the introduction of the appropriate microbes, can produce ethanol, which begins to evaporate into a gas at about 144° F. Thus, with operating temperature within the interior of the pile 10 being preferably in the 150° F. to 160° F. range, ethanol created by the bacteria within the pile 10 will be substantially in the form of an ethanol gas. If genetically engineered bacteria are ultimately utilized in this storage process, substantial amounts of ethanol gas will be created within the pile 10. By introducing a gas into the pipes 28, such as carbon dioxide, that is non-reactive with the ethanol gas and that does not introduce a supply of oxygen into the interior of the pile 10, a gas flow can be created by drawing gases out of the other perforated pipes 29 to extract the ethanol gas from the interior of the pile 10. The ethanol gas can then be cooled to convert the gas into liquid ethanol while the non-reactive gas is re-circulated through the pipes 28, 29 to continue the extraction process. This process can be adapted to the production of other biofuels or biochemicals with the addition of appropriate microbes to the pile and development of appropriate temperature ranges within the interior of the pile 10, as the pipes 28, 29 can be utilized to extract gases or liquids from within the pile 10.

It will be understood that changes in the details, materials, steps and arrangements of parts which have been described and illustrated to explain the nature of the invention will occur to and may be made by those skilled in the art upon a reading of this disclosure within the principles and scope of the invention. The foregoing description illustrates the preferred embodiment of the invention; however, concepts, as based upon the description, may be employed in other embodiments without departing from the scope of the invention.

Having thus described the invention, what is claimed is:

1. A method of storing cellulosic biomass material comprising the steps of:
accumulating cellulosic biomass material into a pile having an average moisture content from about 20% to about 50%, said pile having an outer surface and an interior, wherein oxygen is limited in said pile to allow a generation of heat within said cellulosic biomass material to attain an interior temperature of between about 110° F. and about 175° F. and achieve an anaerobic state within the pile.

2. The method of claim 1 wherein said accumulating step includes the step of:
packing the cellulosic biomass material to increase density of the cellulosic biomass material forming said pile.

3. The method of claim 1 wherein said accumulating step includes the step of:
shaping said pile of cellulosic biomass material so that the outer surface of said pile is substantially rounded without valleys and peaks, said pile of cellulosic biomass material having a base dimension larger than a maximum height dimension.

4. The method of claim 1 further comprising the step of:
controlling the passage of oxygen into said pile of cellulosic biomass material.

5. The method of claim 4 wherein said controlling step includes the step of:
covering said pile of cellulosic biomass material with an impermeable material.

6. The method of claim 1 wherein said cellulosic biomass material includes cellulose and hemicellulose, said method further comprising the step of:
introducing enzymes into said pile to hydrolyze the cellulose and hemicellulose into sugars.

7. The method of claim 7 wherein said introducing step includes the step of incorporating enzyme producing microbes into said pile.

8. The method of claim 1 further comprising the step of:
introducing biochemical producing microbes into said pile.

9. The method of claim 1 further comprising the step of:
mixing cellulosic biomass materials having diverse moisture contents so that the average moisture content is substantially uniformly distributed.

10. The method of claim 9 wherein cellulosic biomass material having less than 25% moisture can be substantially uniformly mixed with biomass material having greater than 50% moisture content such that an average moisture content of said cellulosic biomass material within the pile lies within the range of 25% to 50%.

11. The method of claim 9 further including the step of:
chopping said cellulosic biomass material into substantially randomly sized pieces before said mixing step.

12. The method of claim 4 wherein said controlling step includes the step of creating a crust on the outer surface of said pile.

13. The method of claim 12 wherein said cellulosic biomass material includes corn cobs and said crust is formed of corn husks mixed with said corn cobs.

14. The method of claim 1 further comprising the step of:
monitoring the temperature within the interior of said pile of cellulosic biomass material.

15. The method of claim 14 further comprising the steps of:
injecting air into said pile if said temperature of the interior of said pile is lower than a desired temperature range; and
injecting carbon dioxide into said pile to displace oxygen within the interior of said pile if said temperature of the interior of said pile is higher than a desired temperature range.

16. A method of storing wet cellulosic feedstock material having a moisture content between approximately 20% and approximately 50% for subsequent processing thereof to obtain biochemicals, including biofuels, therefrom, comprising the steps of:
accumulating said cellulosic feedstock material into a pile whereby sufficient oxygen is incorporated into said pile to permit a generation of heat within said pile to attain an interior temperature between about 110° F. and about 175° F. and achieve a substantially anaerobic state within said pile, said pile having an outer surface and an interior; and
maintaining said substantially anaerobic state within said pile.

17. The method of claim 16 wherein said maintaining step includes the step of:
covering said pile of cellulosic feedstock material with an impermeable, flexible material.

18. The method of claim 16 wherein said cellulosic feedstock material includes cellulose and hemicellulose, said method further comprising the step of:
introducing enzymes into said pile to hydrolyze the cellulose and hemicellulose into sugars.

19. The method of claim 18 wherein said introducing step includes the step of incorporating enzyme producing microbes into said pile.

20. The method of claim 16 further comprising the step of:
introducing biochemical producing microbes into said pile.

21. A method of processing wet cellulosic feedstock material having a moisture content between approximately 20% and 50% for subsequent processing thereof to obtain biochemicals, including biofuels, therefrom, comprising the steps of:
accumulating cellulosic feedstock material into a pile wherein oxygen is limited in said pile to allow a generation of heat within said pile to attain an interior temperature of between about 110° F. and about 175° F. and achieve an anaerobic state within said pile, said pile including an outer surface and an interior;
covering said outer surface of said pile with an impermeable material to restrict the flow of air and water into said pile;
injecting a non-reactive gas into said pile to displace biochemical gases generated within said pile; and
collecting the displaced biochemical gases from said pile.

22. The method of claim 21 further comprising the step of:
introducing biochemical producing microbes into said pile.

23. The method of claim 21 further comprising the step of:
placing a conduit system in contact with the cellulosic feedstock material to conduct said injecting and collecting steps.
